(19) United States
(12) Patent Application Publication
(10) Pub. No.: US 2022/0233380 A1
(43) Pub. Date: Jul. 28, 2022

(54) NEGATIVE-PRESSURE DOME

(71) Applicant: FlexSys, Inc., Ann Arbor, MI (US)

(72) Inventors: Sridhar Kota, Ann Arbor, MI (US); Shalini Sarala Kota, Ann Arbor, MI (US)

(21) Appl. No.: 17/580,716

(22) Filed: Jan. 21, 2022

Related U.S. Application Data
(60) Provisional application No. 63/140,298, filed on Jan. 22, 2021.

(51) Int. Cl. A61G 10/02; A61G 10/00

(52) U.S. Cl. CPC A61G 10/023; A61G 10/005

(57) ABSTRACT

A negative-pressure dome includes a transparent domed shield, a flexible skirt attached to the perimeter of the domed shield, a hose attachment port formed on the domed shield; and access openings in at least one of the domed shield and the flexible skirt. The transparent domed shield consists of a clear rigid plastic. The access openings may be at least partially occluded by flexible flaps that are deflectable to increase an open cross-section of the access openings. In a first configuration, the access openings are disposed in the domed shield sized to cover a face and are sized to have a cross-section between 3 cm² and 200 cm². In a second configuration, the access openings are disposed in the flexible skirt and are sized to have a cross-section between 30 cm² and 200 cm², while the domed shield is sized to cover head and chest of a subject.

### TECHNICAL FIELD

The present disclosure relates to protective equipment suited for medical procedures performed on a subject's head, in particular in a subject's mouth.

### BACKGROUND

When a person infected with a contagious condition exhales aerosols, these aerosols may remain suspended in the surrounding atmosphere for an extended period of time. Generally, social distancing and wearing filtering masks mitigate the dangers to other people in indoor settings. In some situations, however, social distancing and mask-wearing are not feasible. For example, dental work requires close proximity between the dentist and the subject. Also, a filtering mask would obstruct access to the oral cavity. There are other situations, in which a subject's mouth or nose need to remain accessible.

### SUMMARY

Accordingly, it is desirable to provide a device that mitigates the presence of pathogen-carrying aerosols in the vicinity of an infected person while also providing access to and visibility of the oral or nasal cavities.

The present disclosure presents negative-pressure dome comprising a transparent domed shield; a flexible skirt attached to the perimeter of the domed shield; a hose attachment port formed on the domed shield; and access openings in at least one of the domed shield and the flexible skirt. This arrangement allows great visibility of a subject's face while keeping any exhaled pathogens from entering the space surrounding the negative-pressure dome. The transparent domed shield preferably consists of a clear rigid plastic to provide undistorted visibility.

The skirt may be transparent, either to allow outside light to illuminate the space covered by the domed shield or to provide additional visual access.

In order to mitigate the required suction power of a vacuum pump creating a negative pressure within the negative-pressure dome, the access openings may be at least partially occluded by flexible flaps that are deflectable to increase an open cross-section of respective access openings when a tool is inserted.

For best optical properties, the domed shield forms a partial ovoid, ellipsoid, or sphere. One or more light sources affixed to the domed shield and directed into the domed shield may also enhance the visibility of features within the negative pressure dome.

In a first configuration, the access openings are disposed in the domed shield and are sized to have a cross-section between 3 cm² and 15 cm².

For constituting a space-saving face covering, the domed shield may have a longitudinal length between 20 cm and 30 cm, a lateral width between 15 cm and 25 cm, and a depth between 2 cm and 10 cm. In this configuration, the domed shield may have a padded rim.

An arcuate wiper operable to wipe an interior surface of the domed shield via an external actuator may enhance the visual properties by removing potential splatter, for example from dental procedures. The wiper may be pivotably mounted to the domed shield with an operating handle extending out of the domed shield.

Alternatively, the access openings may be disposed in the flexible skirt and be sized to have a cross-section between 30 cm² and 200 cm².

This is in particular useful if the domed shield is sized to extend over ahead and chest of a subject. In such a configuration, the domed shield has a longitudinal length between 50 cm and 100 cm, a lateral width between 30 cm and 80 cm, and a depth between 10 cm and 50 cm.

An articulated mount carrying the domed shield keeps the domed shield at such a distance from the subject's body that a working space is created that allows for easy maneuvering of tools.

The articulated mount is attached to the domed shield separately from the hose attachment port so that the hose becomes disposable after every use. In this variation, a hose is in fluid communication with the hose attachment port at a first end of the hose and configured to be connected to a suction port of a vacuum pump at a second end of the hose. The hose is preferably supported by an arm of the articulated mount via a releasable retainer coupling the hose to the arm of the articulated mount.

A filter arrangement at the hose attachment port prevents contamination of the parts downstream from the hose attachment port.

Alternatively or additionally, a filter arrangement disposed at the suction side of the vacuum pump protects the interior of the vacuum pump from contamination.

Further details and benefits of the present disclosure become apparent from the following description of the drawings.

### BRIEF DESCRIPTION OF THE DRAWINGS

In the drawings,

FIG. 1 shows a first configuration of a negative-pressure dome according to the present disclosure;

FIG. 2 shows a partial detail view of the device of FIG. 1;

FIG. 3 shows a cross-sectional view through the device of FIG. 1 along the line III-III;

FIG. 4 shows a detail view of a second configuration of a negative-pressure dome according to the present disclosure;

FIG. 5 shows the device of FIG. 4 in an environment of a dental office; and

FIG. 6 shows a variation of the second configuration of the negative-pressure dome.

The drawings are provided herewith for purely illustrative purposes and are not intended to limit the scope of the present invention.

### DETAILED DESCRIPTION

The present disclosure describes the overall concept of protection against airborne pathogens exhaled by a subject undergoing a facial or oral procedure, such as dental work while ensuring visibility of and access to the target site. The device may also be used for transporting subjects in an ambulance or for subjects in a stationary setting such as in a hospital or a nursing home or home to protect the environment from airborne pathogens in the air exhaled by the subject suffering from or suspected of a contagious respiratory disease.

All discussed configurations and variations have in common that a transparent, preferably clear, domed shield in combination with a skirt and a hose attachment creates an individual negative-pressure dome.

The protection is effective for subjects sitting upright or in a reclined position as customary in dentist chairs. The protection is limited to the individual subject positioned under or behind the negative-pressure dome so that medical or service personnel is free to move around the subject or to leave the room as needed without endangering other individuals.

In this application, the term “negative pressure” defines a pressure lower than the environmental (ambient) atmospheric pressure. Accordingly, the term “negative pressure” indicates a relative pressure compared to the atmosphere, not an absolute pressure: Mathematically subtracting the atmospheric pressure from the pressure inside the negative-pressure dome results in a negative value.

The present disclosure presents two configurations of a negative-pressure dome 10 and 110 with some common and some different features. Where the features differ, features of one of the two configurations may replace the different feature in the other configuration so that this disclosure also encompasses hybrid constructions composed of features from different embodiments. In summary, the two configurations 10 and 110 involve the following features:

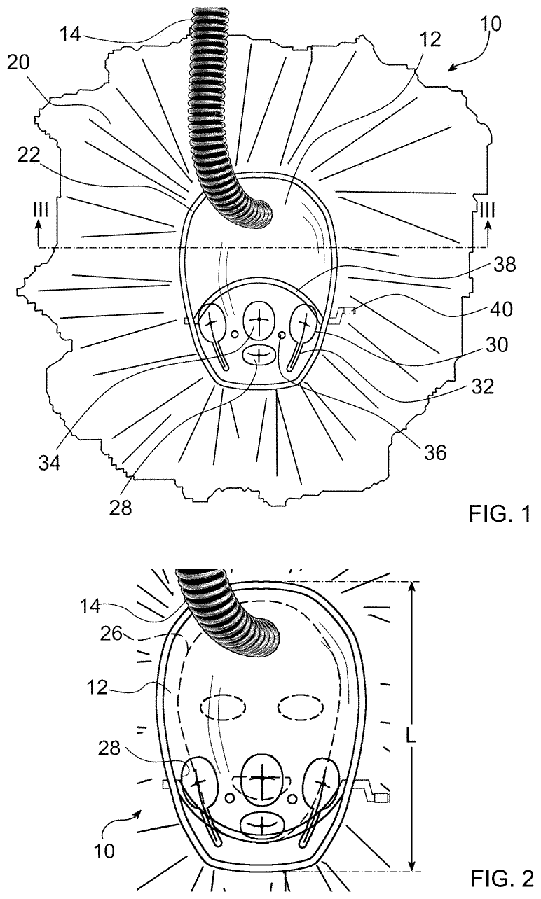

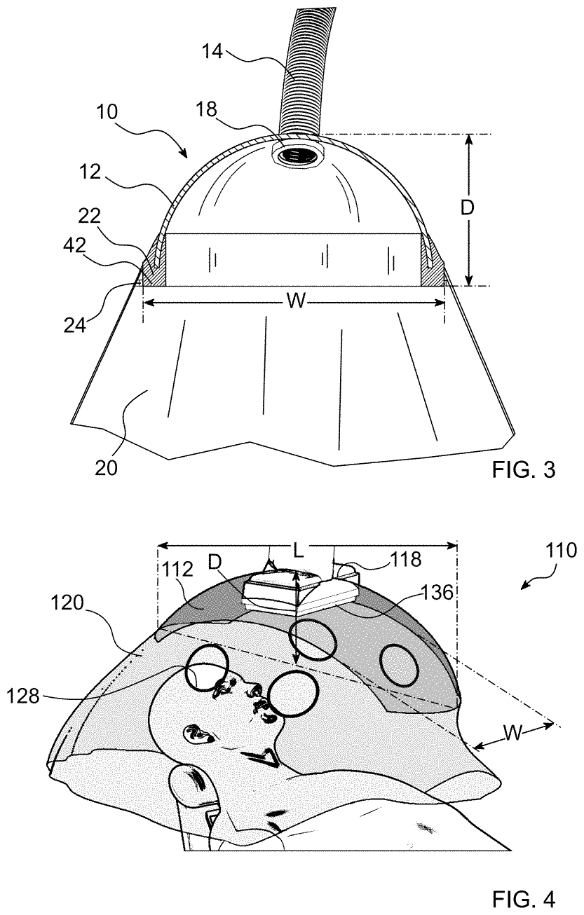

In the first configuration shown in FIGS. 1-3, the negative-pressure dome 10 includes a transparent domed shield 12 attached to a vacuum source (not shown) via a hose 14. The transparent domed shield consists of a clear rigid plastic and is preferably coated with a scratch-resistant clear coating to mitigate potential scratches from tools. The hose 14 is configured to be connected to a vacuum source, such as a vacuum pump equipped with a suitable filter, e.g. a HEPA filter, at or upstream of the suction port of the vacuum source. The vacuum pump creates suction at the unidirectional air flow through the air exchange arrangement within the range of 2,500 liter per minute through 11,000 liter per minute, wherein openings in the flexible skin and surrounding the flexible skin allow for an air flow compensating for the unidirectional air flow through the air exchange arrangement.

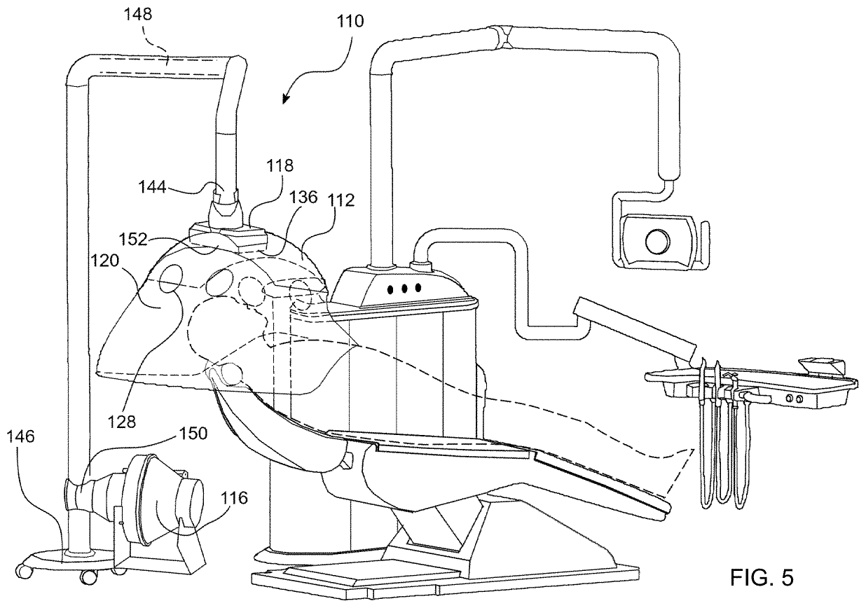

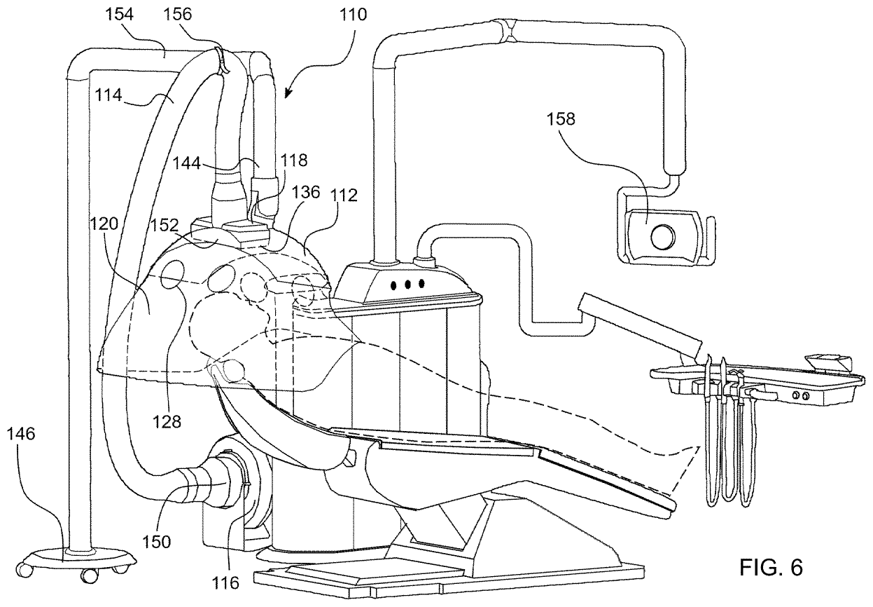

The hose may be a corrugated hose configured to withstand vacuum forces without collapsing. While a vacuum source is not shown in FIGS. 1-3 for the first configuration, it is shown as a compact vacuum pump 116 in FIGS. 5 and 6 in connection with the second configuration 110. The hose 14 is attached to the domed shield 12 via a hose attachment port 18 to establish a fluid communication from the interior space of the domed shield 12 to the hose 14. The domed shield 12 of the first configuration is dimensioned to cover a human face and thus has a longitudinal length L between 20 cm and 40 cm, lateral width W between 15 cm and 30 cm, and a depth D between 2cm and 10 cm

The negative-pressure dome includes a skirt 20 attached to the rim 22 of the domed shield 12. The attachment of the skirt 20 to the rim 22 is preferably achieved by releasable connectors 24 and may, for example, be formed by hook-and-loop connections (known as Velcro®) or snaps distributed along the rim. The domed shield 12 is placed over a subject's face and may be held in place with clips attaching the skirt to the chair, on which the subject rests. While it is possible to leave the skirt 20 hanging freely, such a skirt 20 would need a sufficient weight and stiffness not to collapse from the applied vacuum. In the shown first configuration, it is not crucial that the skirt 20 is transparent because the domed shield 12 provides a wide viewing angle onto the subject's face 26 (shown in FIG. 2). A transparent or clear skirt will, however, enhance the illumination of the subject's face from external light sources. Thus, in addition to transparent flexible films, suitable materials for the skirt 20 are woven, nonwoven, or bonded textiles, including Tyvek®.

The domed shield 12 has access openings 28 for treating the subject with various tools. The access openings have cross-sections between 3 cm² and about 15 cm² and have an elliptical or circular basic shape 30. Optionally, one or more of the access openings may be enlarged by slits 32 extending away from the elliptical or circular basic shape 30 to ease the introduction and movement of tools.

Flexible flaps 34 may be disposed in the access openings 30 to operate as self-closing valves to limit the overall open cross-section in communication with the surroundings. The flexible flaps 34 are deflectable to increase an open cross-section of the access openings 30. The flaps 34 don't need to seal the access openings 30 completely as influx of surrounding air into the interior of the domed shield 12 is part of the operating principle. The suction of the vacuum pump creates a unidirectional air flow through the hose 14 of several hundreds of liters per minute, wherein the access openings 28 in the domed shield 12 and gaps at the edge of the skirt 20 allow for an air flow of surrounding air into the negative-pressure dome.

Vacuum suction is applied through the hose 14 at a sufficient flow rate to draw room air into the domed shield 12 through gaps between the skirt 20 and the surface on which it rests and through the open cross-sections of the access openings 30. The hose 14 leads to a HEPA filter and the suction port of the suction device as mentioned above. The domed shield 12 can be sterilized and reused. The skirt 20, the hose 14, and the HEPA filter may be of single-use and disposable after each use.

Further optional features of the negative-pressure dome 10 of the first configuration include light sources 36 attached to the outside or to the inside of the domed shield 12. The light-sources may be LEDs consuming little energy that can be supplied by small batteries, for example button cell batteries, that do not obstruct the field of vision.

Additionally, especially for implementations involving splatter, a wiper 38 operable from the outside may be installed inside the domed shield 12. The wiper 38 shown is pivotably arranged behind the domed shield 12. an operating handle or crank 40 enables an external operation to swing the wiper up (FIG. 1) or down (FIG. 2) out of the field of vision. The wiper 38 has a curvature adapted to make a line-shaped contact with the interior surface of the domed shield 12 over a range different angular positions so that at least a working area can be cleared of splatter without removing the negative-pressure dome 10.

The rim 22 may be equipped with elastomeric padding 42, for example made of soft silicone or closed-cell foam, that allows surface cleaning and does not trap pathogens in crevices. this may facilitate comfortable support of the domed shield 12 on a subject's head or head rest without cutting into the subject's neck. The padding is not intended to form a seal 42 because environmental air needs to enter the domed shield 12 past the padding 42.

Now referring to the second configuration shown in FIGS. 4-6, a larger domed shield 110 may be more feasible for applications requiring greater movements or larger tools. In the second configuration 110, the domed shield 112 is sized to extend over a subject's head and chest. The domed shield 110, exclusive of the skirt 120 has dimensions in the following ranges: a longitudinal length L between 50 cm and 100 cm, a lateral width W between 30 cm and 80 cm, and a depth D between 10 cm and 50 cm. It is placed at a greater distance from the subject to provide more maneuvering space within the negative-pressure dome 110. As the domed shield 112 is placed at a greater distance from the subject's head than in the first configuration, the skirt 120 is preferably made of a clear transparent material. The domed shield is preferably transparent or clear as well.

Rather than in the domed shield 112, in this configuration, the access openings 128 are disposed in the skirt 120 so that the operator can reach the subject through the access openings 128 or from under the skirt 120. The number, size and location of access openings 128 can be tailored for various procedures as needed. Generally, each access opening 128 has an opening cross-section between 30 cm² and 200 cm² because not only the tip of a tool needs to be inserted, but typically the operator's gloved hand as well.

The skirt 120 around the perimeter of the domed shield 112 has a length dimensioned to rest on the subject's upper torso and to hang down behind the subject's head. If desired, the skirt 120 of the second configuration may be pre-molded into a desired approximate shape with enough flexibility to adapt to the subject's body shape.

To create a suitable working space, the domed shield 112 is suspended or otherwise supported above the subject's face. As shown in FIG. 5, illustrated to resemble a dental operatory, the negative-pressure dome 110 is held by an articulated mount 144 supported by a floor stand 146. The floor stand is only schematically illustrated and is generally designed to provide sufficient counter weight to prevent toppling of the assembly. In a variation, the articulated mount 144 may be mounted to a wall or to the ceiling. The articulated mount allows tilting of the domed shell 112 in all directions and may include a universal joint (also called cardan joint) or a ball joint, especially a hollow ball joint as customary in shower heads.

In a first variation of the second configuration as shown in FIG. 5, the articulated mount 144 includes an interior air flow lumen 148 in fluid communication with the hose attachment port 118 at a first end of the interior air flow lumen 148. The interior air flow lumen leads through the arm of the articulated mount to the suction port 150 of the vacuum pump 116. In the variation shown in FIG. 5, the vacuum pump 116 may be integrated in the floor stand of the articulated mount.

Because the hose attachment port 118 doubles as coupling for the articulated mount 144, the articulation of the articulated mount 144 is constructed to surround the air flow lumen and to allow an unrestricted air flow from the interior of the domed shield 112 to the vacuum pump 116. Further, the hose attachment port 118 includes a filter compartment 152 for a suitable particle filter, such as a HEPA filter.

A light source 136 may be integrated in the housing of the hose attachment port 118 or of the filter compartment 152 and may be powered by wiring extending through the articulated mount 144 or, alternatively, by a battery accommodated at or in the housing of the filter compartment 152 or of the hose attachment port 118.

In another variation shown in FIG. 6, the articulated mount 144 is attached to the domed shield 112 separate from the hose attachment port 118. Accordingly, a separate hose 114 is connected to the hose attachment port and leads to the suction port 150 of the vacuum pump 116. The hose 114 is supported by an arm 154 of the articulated mount 144 via a releasable retainer 156 coupling the hose 114 to the arm 154 of the articulated mount 144. The retainer 156 may be a strap with a snap closure as shown or with a hook-and-loop connection. Alternatively, the retainer 156 may be a resilient Ω-shaped bracket attached to the arm 154 and configured to grip the hose 114 upon insertion. This variation permits the use of a customary arm 154 for the articulated mount 144 of the negative-pressure dome 110 as it is already available for other devices, such as the work light 158, including electric wiring. In all other aspects, the variation of FIG. 6 corresponds to the variation of FIG. 5. The hose 114 is held in place, so it does not interfere with the field of vision.

Both variations have in common that a filter compartment 152 may be disposed upstream of the hose attachment port 118 so that potentially exhaled pathogens are confined to the domed shell 112 and don't enter the air flow lumen 148 or the hose 114, respectively. Additionally or alternatively, the suction port 150 may include a suitable filter, especially if the hose 114 is separate from the articulated mount 144 and is disposed after every use. The suction of the vacuum pump 116 creates a unidirectional air flow through the hose 114 or through the air flow lumen 148 amounting to at least 1000 liters per minute, wherein the access openings 128 in the skirt 120 and gaps at the edge of the skirt 120 allow for an air flow of surrounding air into the negative-pressure dome 110.

In all configurations and variations, the vacuum pump 116 generates a sufficient pressure gradient to draw surrounding air into the negative-pressure dome 10 or 110 and draw the inside the negative-pressure dome 10 or 110 out through a HEPA filter. The domed shield 12 or 112 can be sterilized and reused, while he HEPA filter is intended for single-use. The skirt 120 and the hose 114 of the second configuration 110 may be reused, or, if cleaning the skirt 120 is considered too time-consuming, the skirt 120 may also be disposable. In the first configuration 10, in which the hose 14 is positioned upstream of the filter, the hose 14 is intended for single use only and may be disposed after each use.

While the above description pertains to the preferred embodiments of the present invention, the invention is susceptible to modification, variation and change without departing from the proper scope and fair meaning of the accompanying claims.

What is claimed is:

1. A negative-pressure dome comprising:
a transparent domed shield;
a flexible skirt attached to the perimeter of the domed shield;
a hose attachment port formed on the domed shield; and
access openings in at least one of the domed shield and the flexible skirt.

2. The negative-pressure dome according to claim 1, wherein the transparent domed shield consists of a clear rigid plastic.

3. The negative-pressure dome according to claim 1, wherein the skirt is transparent.

4. The negative-pressure dome according to claim 1, wherein the access openings are at least partially occluded by flexible flaps that are deflectable to increase an open cross-section of the access openings.

5. The negative-pressure dome according to claim 1, wherein the domed shield forms a partial ovoid, ellipsoid, or sphere.

6. The negative-pressure dome according to claim 1, further comprising one or more light sources affixed to the domed shield and directed into the domed shield.

7. The negative-pressure dome according to claim 1, wherein the access openings are disposed in the domed shield and are sized to have a cross-section between 3 cm² and 15 cm².

8. The negative-pressure dome according to claim 1, wherein the domed shield has a longitudinal length between 20 cm and 30 cm, a lateral width between 15 cm and 25 cm, and a depth between 2 cm and 10 cm.

9. The negative-pressure dome according to claim 8, wherein the domed shield has a padded rim.

10. The negative-pressure dome according to claim 1, further comprising an arcuate wiper operable to wipe an interior surface of the domed shield via an external actuator.

11. The negative-pressure dome according to claim 10, wherein the wiper is pivotably mounted to the domed shield with an operating handle extending out of the domed shield.

12. The negative-pressure dome according to claim 1, wherein access openings are disposed in the flexible skirt and are sized to have a cross-section between 30 cm² and 200 cm².

13. The negative-pressure dome according to claim 1, wherein the domed shield has a longitudinal length between 50 cm and 100 cm, a lateral width between 30 cm and 80 cm, and a depth between 10 cm and 50 cm.

14. The negative-pressure dome according to claim 1, further comprising an articulated mount carrying the domed shield.

15. The negative-pressure dome according to claim 14, wherein the articulated mount is attached to the domed shield separately from the hose attachment port.

16. The negative-pressure dome according to claim 15, further comprising a hose in fluid communication with the hose attachment port at a first end of the hose and configured to be connected to a suction port of a vacuum pump at a second end of the hose.

17. The negative-pressure dome according to claim 16, wherein the hose is supported by an arm of the articulated mount via a releasable retainer coupling the hose to the arm of the articulated mount.

18. The negative-pressure dome according to claim 1, further comprising a filter arrangement at the hose attachment port.

19. The negative-pressure dome according to claim 1, further comprising a vacuum pump and a hose configured to be attached at the hose attachment port with a filter arrangement disposed at a suction side of the vacuum pump.
